(19) United States
(12) Patent Application Publication
(10) Pub. No.: US 2007/0003559 A1
(43) Pub. Date: Jan. 4, 2007

(54) METHODS OF DETERMINING PHARMACOKINETICS OF TARGETED THERAPIES

(75) Inventors: Kiran Khandke, Nanuet, NY (US); Nitin K. Damle, Upper Saddle Rive, NJ (US); Erwin R. Boghaert, Monroe, NY (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/427,650

(22) Filed: Jun. 29, 2006

Related U.S. Application Data
(60) Provisional application No. 60/695,419, filed on Jul. 1, 2005.

(51) Int. Cl. A61K 39/395; G01N 33/53

(52) U.S. Cl. 424/155.1; 424/178.1; 435/7.1

(57) ABSTRACT

Methods for determining pharmacokinetics of targeted therapies.

### RELATED APPLICATIONS

Priority is claimed to U.S. Provisional Patent Application No. 60/695,419, filed on Jul. 1, 2005, which is incorporated by reference in its entirety herein.

### FIELD OF THE INVENTION

The present invention generally relates to methods for determining pharmacokinetic properties of targeted therapies (e.g., immunoconjugates) using mass-sensing techniques.

### BACKGROUND OF THE INVENTION

Synthetic and natural macromolecules have become established therapeutics in cancer treatment. Antibodies have proven clinical efficacy when administered as a naked or unconjugated antibody or as an antibody/drug conjugate. According to the latter approach, a therapeutic agent is coupled to an antibody with binding specificity for a defined target cell population. Therapeutic agents that have been conjugated to monoclonal antibodies include cytotoxins, biological response modifiers, enzymes (e.g., ribonucleases), apoptosis-inducing proteins and peptides, and radioisotopes.

Antibody-mediated drug delivery to tumor cells augments drug efficacy by minimizing its uptake in normal tissues. See e.g., Reff et al. (2002) Cancer Control 9:152-66; Sievers (2000) Cancer Chemother. Pharmacol. 46 Suppl:S18-22; Goldenberg (2001) Crit. Rev. Oncol. Hematol. 39:195-201. MYLOTARG® (gemtuzumab ozogamicin) is a commercially available targeted immunotherapy that works according to this principle and which is approved for the treatment of acute myeloid leukemia in elderly patients. See Sievers et al. (1999) Blood 93: 3678-3684. In this case, the targeting molecule is an anti-CD33 monoclonal antibody that is conjugated to calicheamicin. Additional examples include ibritumomab tiuxetan (ZEVALIN®) and tositumomab (BEXXAR®), which are radiolabeled anti-CD20 antibodies. See Dillman, Clin. Exp. Med., 2006, 6(1):1-12.

Despite progress in developing new antibody-targeted therapies, the physiological characteristics conferring a favorable therapeutic index in the clinic are not well understood. Simple biochemical assays (e.g., the affinity of antibody for its antigen) do not necessarily predict efficacy. See Graff & Wittrup, Cancer Res., 2003, 63(6):1288-1296. Biological parameters in vivo such as circulation half-life, tissue distribution rates, and rate of conjugate degradation may be more helpful in comparing the potential therapeutic efficacy of these molecules. However, preclinical experiments designed to assess these parameters are difficult because they typically require large numbers of experimental animals and radiolabeling of the conjugate.

To address the need for methods of predicting clinical efficacy, the present invention provides plasmon resonance assays for pharmacokinetic characterization of targeted therapies following their administration to a subject. The assays disclosed herein accurately and reproducibly detect amounts of targeting molecule and targeting molecule/drug conjugate in a single, minimal volume sample. Based upon this determination, the circulation half-life of targeting molecule/drug conjugate, rates of conjugate degradation, and linker stability can be monitored in a subject.

### SUMMARY OF THE INVENTION

The present invention provides methods of determining an amount of targeting molecule and an amount of targeting molecule/drug conjugate in a sample. In a representative embodiment of the invention, the method comprises the steps of: (a) providing a solid support comprising a surface to which a target is immobilized; (b) providing a sample comprising a plurality of targeting molecule/drug conjugates; (c) contacting the sample with the target immobilized to the surface of the solid support; (d) detecting formation at the surface of the solid support of a first binding complex of (i) the targeting molecule and (ii) the target at the surface of the solid support, wherein formation of the first binding complex causes a first measurable change in mass property of the solid support indicating an amount of targeting molecule in the sample; (e) contacting the first binding complex with a drug binding agent that specifically binds the drug of the targeting molecule/drug conjugate; and (f) detecting formation at the surface of the solid support of a second binding complex of (i) the drug binding agent and (ii) the first binding complex, wherein formation of the second binding complex causes a second measurable change in mass property of the solid support indicating an amount of targeting molecule/drug conjugate in the sample.

Methods of determining an amount of targeting molecule/drug conjugate in a sample can also comprise the steps of: (a) providing a solid support comprising a surface to which a first binding complex is immobilized, wherein the binding complex comprises (i) a target and (ii) a targeting molecule/drug conjugate bound to the target; (b) contacting a drug binding agent that specifically binds the drug of the targeting molecule/drug conjugate with the first binding complex immobilized at the surface of the solid support; and (c) detecting formation of a second binding complex of (i) the drug binding agent and (ii) the first binding complex at the surface of the solid support, wherein formation of the complex causes a measurable change in mass property of the solid support indicating an amount of targeting molecule/drug conjugate in the sample.

In another aspect of the invention, methods of determining an average amount of drug loading per targeting molecule are provided. For example, a method of determining drug loading of targeting molecule/drug conjugates in a sample can comprise the steps of: (a) providing a solid support to which targeting molecule/drug conjugates of a sample are bound; (b) determining an amount of drug in the sample by measuring a change in mass property of a solid support upon binding of a drug binding agent, which specifically binds the drug of the targeting molecule/drug conjugate, to the targeting molecule/drug conjugates at the surface of the solid support; and (c) calculating an average amount of drug per targeting molecule/drug conjugate by dividing the amount of drug of (b) by an amount of targeting molecule in the sample.

### BRIEF DESCRIPTION OF THE DRAWINGS

FIG. 1 shows a sensorgram of a sandwich detection method. In the first phase of the curve (between arrow 1 and 2), the sample of antibody/calicheamicin conjugate was run over the immobilized antigen. A second phase (between arrow 3 and 4) was initiated by adding an anti-calicheamicin antibody. Response 1 indicates mass addition proportionate to the concentration of antibody in the sample, and response 2 is proportionate to the amount of calicheamicin in the antibody/calicheamicin conjugate. RU, resonance units; gray circles, washing period.

FIGS. 2A-2B show the correlation between the amount of antibody or antibody/drug conjugate and the concentration of standard samples. FIG. 2A is a sensorgram showing resonance units as a function of time for each of the indicated concentrations (ng/ml) of hP67.6-AcBut-CalichDMH. FIG. 2B is a line graph showing resonance units as a function of concentration of hP67.6-AcBut-CalichDMH+anti-calicheamicin antibody (black filled circle), hP67.6-AcBut-CalichDMH (gray filled circle), and anti-calicheamicin antibody (open circle).

FIGS. 3A-3C show plasma concentrations of hP67.6-AcBut-CalichDMH determined using a sandwich detection method as described in Examples 3 and 4. Each animal received antibody/drug conjugate for a total dose of 3 μg of calicheamicin. The dose of antibody/drug conjugate expressed in mg/kg is indicated. Solid lines, animals bearing CD22-positive Ramos tumors; dotted lines, tumor-free mice.

FIG. 3A shows response 1, i.e., binding of hP67.6 and hP67.6-AcBut-CalichDMH, to CD33 antigen immobilized on a CM5 chip.

FIG. 3B shows response 2, i.e., binding of anti-calicheamicin to hP67.6-AcBut-CalichDMH already bound to CD33 immobilized on a CM5 chip. The kinetics of hP67.6-AcBut-CalichDMH in plasma are similar in tumor-bearing and tumor-free animals.

FIG. 3C shows the ratio of response 2 relative to response 1. The declining concentration of antibody/drug conjugate as a fraction of the concentration of the antibody moiety of the antibody/drug conjugate indicates the preferential clearance of conjugated versus unconjugated antibody.

FIG. 4 is a line graph showing resonance units as a function of concentration of G5/44-AcBut-CalichDMH (inotuzumab ozogamicin)+anti-calicheamicin antibody (black filled circle), G5/44-AcBut-CalichDMH (gray filled circle), and anti-calicheamicin antibody (open circle).

FIGS. 5A-5C show plasma concentrations of G5/44-AcBut-CalichDMH determined using a sandwich detection method as described in Examples 3 and 5. G5/44 anti-CD22 antibody was loaded with 72 μg calicheamicin per mg antibody, and each animal received antibody/drug conjugate for a total dose of 3 μg of calicheamicin. Solid lines, animals bearing CD22-positive Ramos tumors; dotted lines, tumor-free mice.

FIG. 5A shows response 1, i.e., binding of G5/44 and G5/44-AcBut-CalichDMH to CD22 antigen immobilized on a CM5 chip.

FIG. 5B shows response 2, i.e., binding of anti-calicheamicin to G5/44-AcBut-CalichDMH already bound to CD22 immobilized on a CM5 chip. The presence of the CD22-positive Ramos tumor (solid lines) decreases the average concentration of G5/44 antibody and G5/44-AcBut-CalichDMH conjugates in plasma.

FIG. 5C shows the ratio of response 2 relative to response 1. The declining concentration of antibody/drug conjugate as a fraction of the concentration of the antibody moiety of the antibody/drug conjugate indicates the preferential clearance of conjugated versus unconjugated antibody. Removal of calicheamicin from the antibody was not influenced by the presence of the CD22-positive Ramos tumor.

### DETAILED DESCRIPTION OF THE INVENTION

The present invention provides methods of characterizing samples comprising compositions for targeted therapy, ie., a targeting molecule conjugated either directly or indirectly to a drug. Samples containing targeting molecule/drug conjugates may include some proportion of the constituent parts (ie., unconjugated targeting molecule and free drug), for example, as a result of incomplete conjugation, degradation of the conjugate, etc. In general, the unconjugated targeting molecule and free drug each have limited efficacy and may contribute to patient toxicity. Accordingly, for monitoring progress in patients receiving targeted therapies, drug loading and the concentration of targeting molecule/drug conjugate (rather than the constituent parts) is important. The disclosed methods provide for such determination, which can be used to assess pharmacokinetic parameters of a targeting molecule/drug conjugate, such as absorption, distribution, metabolism, and excretion, following administration to a subject.

As compared to prior methods, the present disclosure describes use of a mass-sensing technique to detect targeting molecule/drug conjugates, wherein such conjugates are labile. The concentration of targeting molecule/drug conjugates can be accurately determined in serum and/or at the targeting site to assess circulation half-life, linker stability, and an amount of drug that is delivered to the targeting site. A single, low-volume sample may be used to sequentially perform multiple detecting steps in a same sample, which enables calculation of drug loading on the targeting molecule/drug conjugate.

I. Targeting Molecule/Drug Conjugates

Targeting molecules that may be used in the disclosed methods include any molecule that shows specific binding to a target. Specific binding refers to an affinity between two molecules which results in preferential binding in a heterogeneous sample. Binding is generally characterized by an affinity of at least about 10⁻⁷ M or higher, such as at least about 10⁻⁸ M or higher, or at least about 10⁻⁹ M or higher, or at least about 10⁻¹¹ M or higher, or at least about 10⁻¹² M or higher.

Targeting molecules also include any molecule that, following administration to a subject, selectively binds to cells expressing the target. The term targeting refers to the preferential movement and/or accumulation in vivo of a molecule at a target site (e.g., cells or tissues) as compared to a control site. A target site comprises cells expressing a target, i.e., an intended site for accumulation of the targeting molecule or targeting molecule/drug conjugate. A control site comprises cells that substantially lack expression of the target and which therefore substantially lack binding and/or accumulation of an administered targeting molecule or targeting molecule/drug conjugate. Selective binding generally refers to a preferential localization of a targeting molecule/drug conjugate such that an amount of targeting molecule at a target site is about 2-fold greater than an amount of targeting molecule at a control site, or about 5-fold greater, or about 10-fold greater or more.

Representative targeting molecules include antibodies, proteins, peptides, peptide mimetics, peptide nucleic acids (PNAs), oligonucleotides, ligands, lectins, and any other molecules that specifically and/or selectively bind to a target.

Targets bound by targeting molecules are generally associated with a disease state, a disease susceptible state, or a condition requiring treatment. Representative targets include antigens, haptens, proteins, peptides, receptors, oligonucleotides, carbohydrates, and any other molecules expressed at elevated levels by cells of a target site. A target is preferably present at the cellular surface or otherwise accessible to targeting molecules. A target site may be localized, such as in a solid tumor, or non-localized as in hematological malignancies. For example, a target site can comprise cells expressing tumor-associated antigens (TAA), antigens expressed on other malignant cells, immune cells contributing to inflammation, allergy, autoimmunity, etc.

In one aspect of the invention, the targeting molecule is an antibody and the invention relates to characterizing samples comprising immunoconjugates, i.e., antibody/drug conjugates. The antibody moiety of an antibody/drug conjugate can comprise any type of antibody, including for example, antibodies having tetrameric structure (e.g., similar to naturally occurring antibodies), or any other structure having at least one immunoglobulin light chain variable region or at least one immunoglobulin heavy chain region, or antigen-binding fragments thereof (e.g., Fab, modified Fab, Fab′, F(ab′)₂ or Fv fragments). The disclosed methods may also be used to characterize conjugates prepared using chimeric antibodies, humanized antibodies, diabodies, single chain antibodies, tretravalent antibodies, and/or multispecific antibodies (e.g., bispecific antibodies).

For preparation of targeted anti-cancer therapies, tumor-associated antigens have been identified that specifically bind to cancer cells from solid tumors, such as squamous/adenomatous lung carcinoma (non-small-cell lung carcinoma), invasive breast carcinoma, colorectal carcinoma, gastric carcinoma, squamous cervical carcinoma, invasive endometrial adenocarcinoma, invasive pancreas carcinoma, ovarian carcinoma, squamous vesical carcinoma, and choriocarcinoma. Antigens for targeted therapy of hematologic malignancies may also be useful drug targets, for example, for the treatment of lymphomas and leukemias, such as including but not limited to low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia, chronic leukocytic leukemia, acute myelogenous leukemia, acute lymphoblastic leukemia, chronic lymphocytic leukemia, chronic myelogenous leukemia, lymphoblastic leukemia, lymphocytic leukemia, monocytic leukemia, myelogenous leukemia, and promyelocytic leukemia.

Representative antibodies that may be used to prepare antibody/drug conjugates for targeted therapy include anti-5T4 antibodies, anti-CD19 antibodies, anti-CD20 antibodies (e.g., RITUXAN®, ZEVALIN®, BEXXAR®), anti-CD22 antibodies, anti-CD33 antibodies (e.g., MYLOTARG®), anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003), hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®), and anti-fibrin antibodies (e.g., MH-1).

As used herein, a drug refers to refers to any substance having biological or detectable activity, for example, therapeutic agents, binding agents, etc., as well as prodrugs, which are metabolized to an active agent in vivo. The term drug also includes drug derivates, wherein a drug has been functionalized to enable conjugation with a targeting molecule.

The drug may be bound to the targeting molecule either directly or indirectly, but the linkage is such that it is compatible with preserving the therapeutic effect of the drug moiety. The linker may be stable or hydrolyzable, and any suitable technique for linking the drug to the antibody may be used. For example, hydrazides and other nucleophiles may be conjugated to the aldehydes generated by oxidation of the carbohydrates that naturally occur on antibodies. Hydrazone-containing conjugates can be made with introduced carbonyl groups that provide the desired drug-release properties. Conjugates can also be made with a linker that has a disulfide at one end, an alkyl chain in the middle, and a hydrazine derivative at the other end. Other representative linkers are thiol-reactive linkers such as esters, amides, and acetals/ketals, and pH sensitive linkers, such as cis-aconitates, which have a carboxylic acid juxtaposed to an amide bond. Linkers may also include solubilizing agents such as PEG to limit aggregation of the targeting molecule/drug conjugates. Peptdie linkers may also be used.

Representative drugs include anti-cancer agents, such as cytotoxic agents, chemotherapeutic agents, immunomodulatory agents, anti-angiogenic agents, anti-proliferative agents, pro-apoptotic agents, enzymes, and bioactive proteins. A drug may also comprise a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. Therapeutic agents may be prepared as pharmaceutically acceptable salts, acids or derivatives of any of the above. In addition, conjugates can be made using secondary carriers as the cytotoxic agent, such as liposomes or polymers, for example.

The term cytotoxic agent generally refers to an agent that inhibits or prevents the function of cells and/or results in destruction of cells. Representative cytotoxic agents include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins. For example, cytotoxic agents include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

In particular aspects of the invention, the targeting molecule/drug conjugates characterized using the disclosed methods comprise an antibiotic drug moiety such as a calicheamicin, also called the LL-E33288 complex, for example, gamma-calicheamicin or a less potent derivative, N-acetyl gamma calicheamicin. See U.S. Pat. No. 4,970,198. Additional examples of calicheamicins suitable for use in targeting molecule/drug candidates are disclosed in U.S. Pat. Nos. 4,671,958; 5,053,394; 5,037,651; 5,079,233; and 5,108,912; which are each incorporated herein in their entirety. Disulfide analogs of calicheamicin can also be used, for example, analogs described in U.S. Pat. Nos. 5,606,040 and 5,770,710, which are each incorporated herein in their entirety. Representative techniques for preparation of antibody/calicheamicin conjugates as set forth in Example 1 are described in U.S. Pat. Nos. 5,712,374; 5,714,586; 5,773,001; and 5,877,296; U.S. Publication Nos. 2004-0082764-A1 and 2006-0002942-A1; and PCT Publication No. WP 2005/089809; which are each incorporated herein in their entirety.

Immunomodulatory agents that may be used to prepare targeting molecule/drug conjugates include anti-hormones that block hormone action on tumors and immunosuppressive agents that suppress cytokine production, downregulate self-antigen expression, or mask MHC antigens. Representative anti-hormones include anti-estrogens including for example tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Representative immunosuppressive agents include 2-amino6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFα antibodies, anti-IL2 antibodies), streptokinase, TGFβ, rapamycin, T-cell receptor, T-cell receptor fragments, and T cell receptor antibodies.

Representative anti-angiogenic agents include inhibitors of blood vessel formation, for example, farnesyltransferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleukin-24, thrombospondin, metallospondin proteins, class I interferons, interleukin 12, protamine, angiostatin, laminin, endostatin, and prolactin fragments.

Anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, friedelane, dammarane, cucurbitacin, and limonoid triterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Triapine)), apoptin (viral protein 3—VP3 from chicken aneamia virus), inhibitors of Bcl-2 and Bcl-X(L), TNF-alpha, FAS ligand, TNF-related apoptosis-inducing ligand (TRAIL/Apo2L), activators of TNF-alpha/FAS ligand/TNF-related apoptosis-inducing ligand (TRAIL/Apo2L) signaling, and inhibitors of PI3K-Akt survival pathway signaling (e.g., UCN-01 and geldanamycin).

Representative chemotherapeutic agents include alkylating agents such as thiotepa and cyclosphosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlornaphazine, cholophosphamide, estramustine, ifosfamide, mechiorethamine, mechiorethamine oxide hydrochloride, melphalan, novembichin, phenesterine, prednimustine, trofosfarnide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicins, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-EU; androgens such as calusterone, dromostanolone propionate, epitiostanol, mepitiostane, testolactone; anti-adrenal such as aminoglutethimide, mitotane, trilostane; folic acid replenisher such as frolinic acid; aceglatone; aldophospharnide glycoside; arninolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2′-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g. paclitaxel (TAXOL®, Bristol-Myers Squibb Oncology of Princeton, N.J.) and doxetaxel (TAXOTERE®, Rhone-Poulenc Rorer of Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aininopterin; xeloda; ibandronate; CPT-11; topoisomerase inhibitor RFS 2000; difluoromethylomithine (DMFO); retinoic acid; esperamicins; and capecitabine.

Additional therapeutic agents that may be conjugated to targeting molecules and characterized using the methods disclosed herein include photosensitizing agents (U.S. Patent Publication No. 2002/0197262 and U.S. Pat. No. 5,952,329) for photodynamic therapy; magnetic particles for thermotherapy (U.S. Patent Publication No. 2003/0032995); binding agents, such as peptides, ligands, cell adhesion ligands, etc., and prodrugs such as phosphate-containing prodrugs, thiophosphate-containing prodrugs, sulfate containing prodrugs, peptide containing prodrugs, β-lactam-containing prodrugs, substituted phenoxyacetamide-containing prodrugs or substituted phenylacetamide-containing prodrugs, 5-fluorocytosine and other 5-fluorouridine prodrugs that may be converted to the more active cytotoxic free drug.

II. Pharmacokinetics of Targeting Molecule/Drug Conjugates

The present invention provides methods of determining drug loading of a targeting molecule, for example, to determine whether the conjugation reaction achieved a level of drug loading which comprises an effective dose, i.e., an amount of targeting molecule/drug conjugate sufficient to elicit a desired biological response, and to maintain batch-to-batch consistency of commercially manufactured targeting molecule/drug conjugates. To assess drug release or stability of targeting molecule/drug conjugates, drug loading may also be assessed following administration to a patient, for example, using a blood sample from the patient.

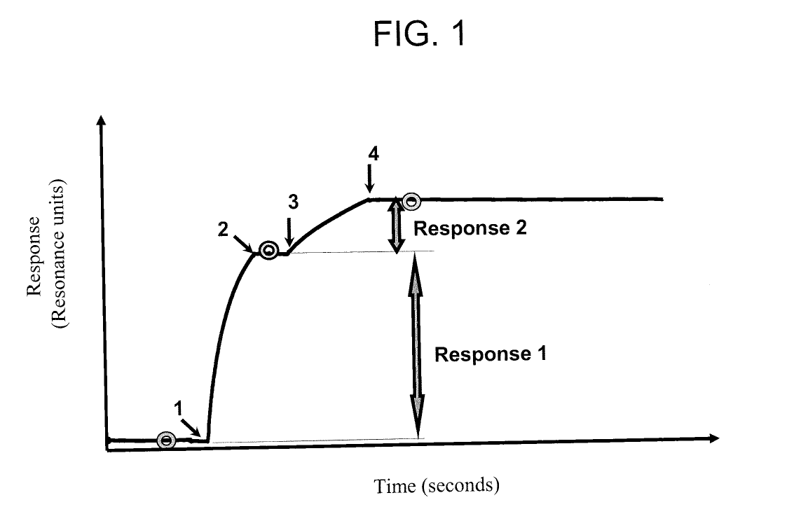

As disclosed herein, an amount of targeting molecule/drug conjugate may be calculated from the separate determinations of (i) an amount of targeting molecule and (ii) an amount of targeting molecule/drug conjugate in the same sample. Steps (i) and (ii) are described herein below more fully under subheadings II.A and II.B, respectively. See also FIG. 1. Briefly, the method includes measurement of two consecutive responses. A first response determines the number of resonance units after contacting a sample that contains the targeting molecule/drug conjugates over a mass sensing device, such as a BIACORE® chip, with immobilized target recognized by the targeting molecule of the conjugate. This response is proportional to the sum of the free (unconjugated) and conjugated targeting molecule in the sample. A second response is obtained after sequentially contacting a drug binding agent with the conjugated and unconjugated targeting molecules bound to the immobilized target on the same mass sensing device. This second response is proportional to the amount of drug present as targeting molecule/drug conjugates in the sample.

In accordance with the disclosed methods, any suitable mass-sensing technique may be used. Representative techniques known in the art include piezoelectric, optical, thermo-optical, surface acoustic wave (SAW) methods, as well as electrochemical methods, such as potentiometric, voltametric, conductometric, amperometric and capacitance methods.

Optical methods that may be used include methods for detecting mass surface concentration (or refractive index), such as reflection-optical methods, including both internal and external reflection methods, e.g., ellipsometry and evanescent wave spectroscopy (EWS), the latter including surface plasmon resonance (SPR), Brewster angle refractometry, critical angle refractometry, frustrated total reflection (FTR), evanescent wave ellipsometry, scattered total internal reflection (STIR), optical wave guide sensors, evanescent wave based imaging, such as critical angle resolved imaging, Brewster angle resolved imaging, SPR angle resolved imaging, etc., as well as methods based on evanescent fluorescence (TIRF) and phosphorescence.

For example, to estimate the equilibrium constant of a targeting molecule in a sample, the following mass-sensing technique may be used. First, a concentration series (e.g., 0, 100, 200, 300, 400, 500, 600, 700, 800, 900, and 1000 ng/ml) of the targeting molecule is prepared and sequentially injected into a biosensor having a sensor chip operatively associated therewith, wherein the sensor chip has a reference sensing surface and at least one sensing surface with immobilized target. The relative responses at steady-state binding levels for each targeting molecule concentration are measured. Because of bulk-refractive index contributions from solvent additives in the biosensor's running buffer, a correction factor may be calculated (via known calibration procedures) and applied to give corrected relative responses. The corrected relative responses for each targeting molecule concentration are then mathematically evaluated as is appreciated by those skilled in the art to estimate the equilibrium constant of the targeting molecule.

In a particular aspect of the invention, the mass sensing technique is surface plasmon resonance, which may be performed using a BIACORE® instrument (Biacore AB of Uppsala, Sweden). The apparatus and theoretical background are described in Jonsson et al., BioTechniques, 1991, 11:620-627. This technique involves immobilizing a first binding partner of a binding pair to a sensor chip, contacting the sensor chip with a sample containing a second binding partner of the binding pair, and then measuring a resultant change in the surface optical characteristics of the sensor chip.

In general, a solid support comprises a hydrogel matrix coating coupled to the top surface of the solid support, wherein the hydrogel matrix coating has a plurality of functional groups. For use with a BIACORE® instrument, the solid support is preferably in the form a sensor chip, wherein the sensor chip has a free electron metal interposed between the hydrogel matrix and the top surface. Suitable free electron metals for this purpose include copper, silver, aluminum and gold.

In a particular aspect of the invention, the method may comprise the steps of: (a) providing a solid support comprising a surface to which a target is immobilized; (b) providing a sample comprising a plurality of targeting molecule/drug conjugates; (c) contacting the sample with the target immobilized to the surface of the solid support; (d) detecting formation at the surface of the solid support of a first binding complex of (i) the targeting molecule and (ii) the target at the surface of the solid support, wherein formation of the first binding complex causes a first measurable change in mass property of the solid support indicating an amount of targeting molecule in the sample; (e) contacting the first binding complex with an anti-drug antibody or drug-binding fragment thereof; and (f) detecting formation at the surface of the solid support of a second binding complex of (i) the anti-drug antibody or drug-binding fragment thereof and (ii) the first binding complex, wherein formation of the second binding complex causes a second measurable change in mass property of the solid support indicating an amount of targeting molecule/drug conjugate in the sample.

In another aspect of the invention, a method of determining an average amount of drug loading per antibody in a sample of targeting molecule/drug conjugates can comprise the steps of: (a) providing a solid support to which targeting molecule/drug conjugates of a sample are bound; (b) determining an amount of drug in the sample by measuring a change in mass property of a solid support upon binding of an anti-drug antibody or drug-binding fragment thereof to the targeting molecule/drug conjugates at the surface of the solid support; and (c) calculating an average amount of drug per targeting molecule/drug conjugate by dividing the amount of drug of (b) by an amount of targeting molecule in the sample. When considered as a function of time following administration to a subject, this method is useful for assessing circulation half-life of a targeting molecule/drug conjugate and linker stability.

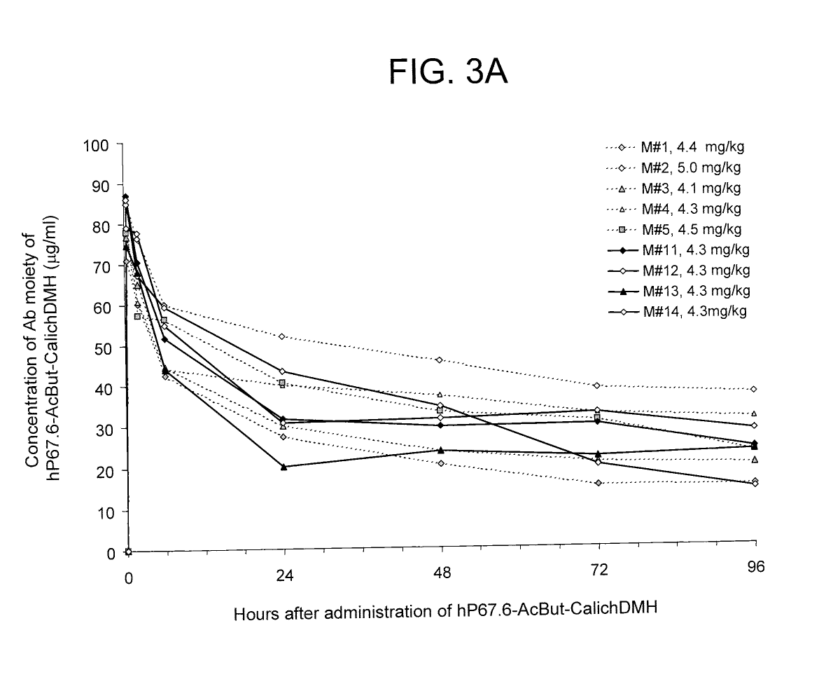

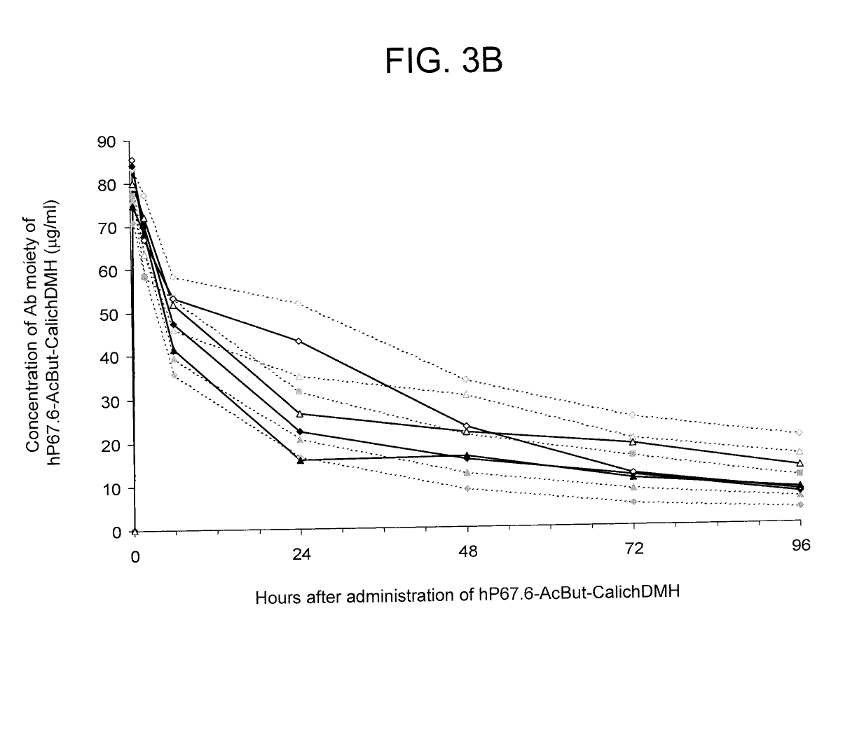

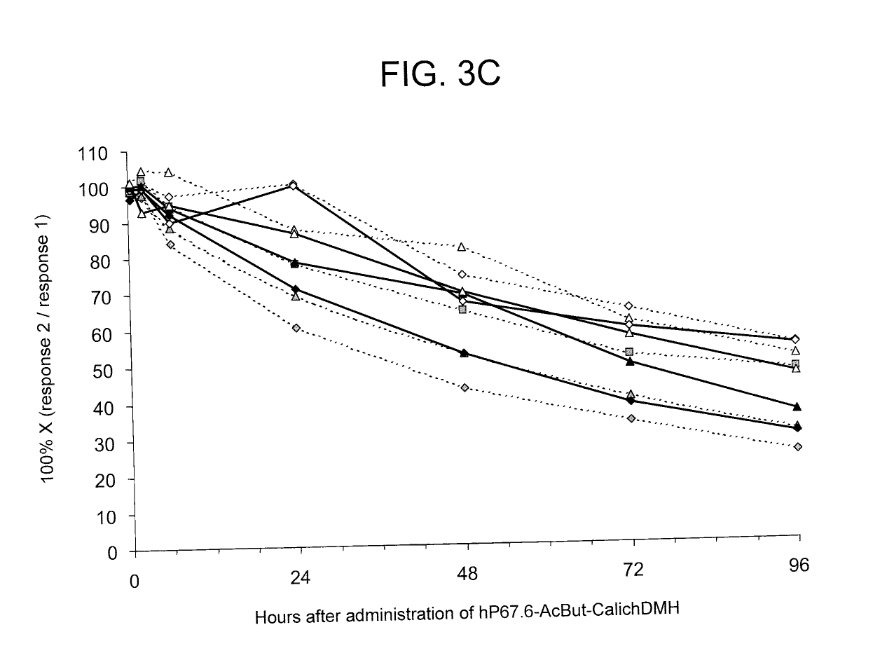

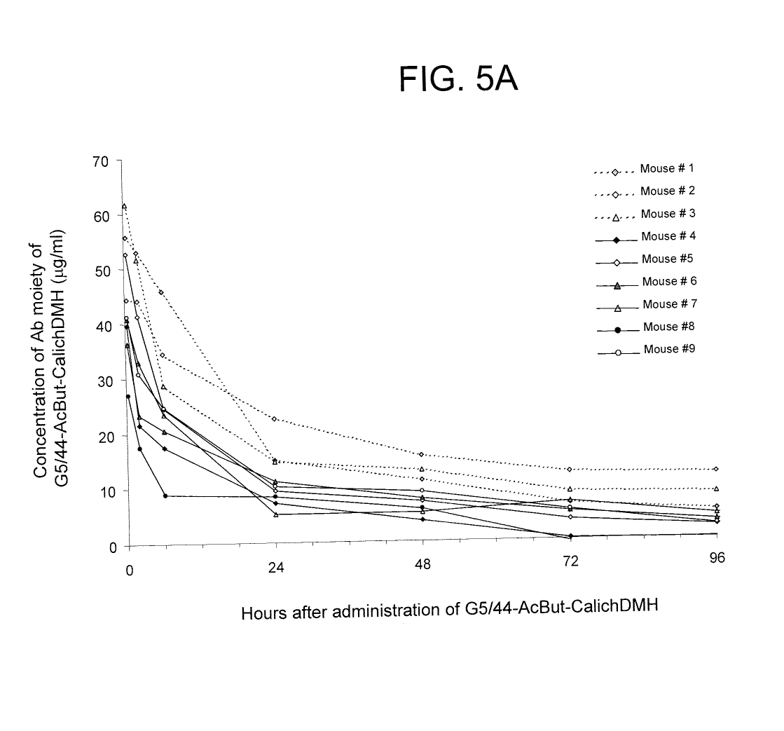

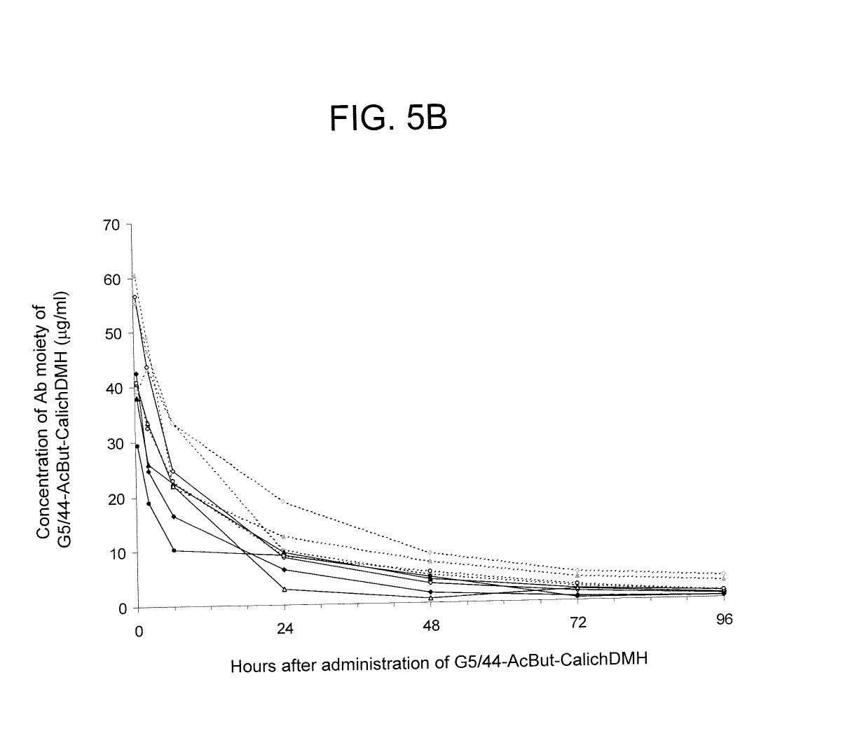

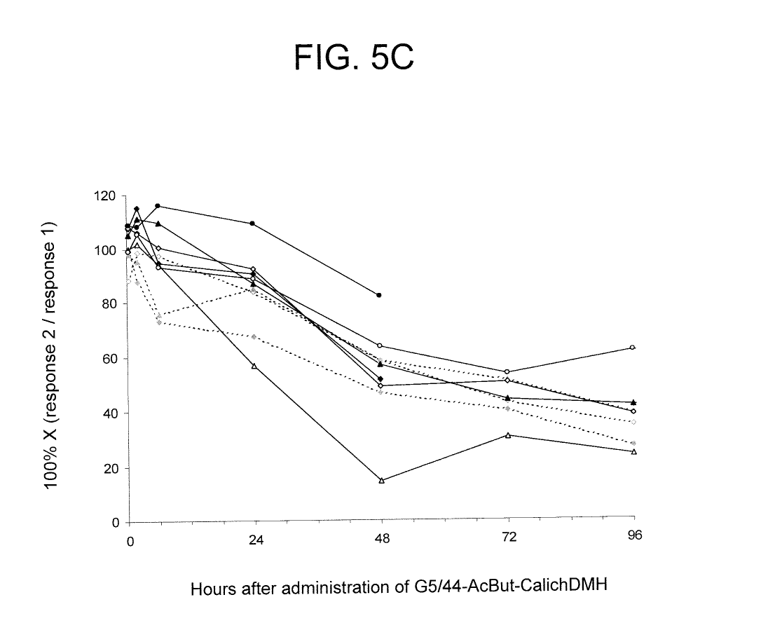

Using the disclosed methods, targeting molecule/drug conjugates were detected in serum samples at a level of 100 to 1,000 ng/ml targeting molecule. As described in Examples 4 and 5, PK values of targeting molecule/drug conjugates were reproducibly determined in individual samples. The presence of a tumor expressing a target reduced the circulation half-life of a targeting molecule/drug conjugate with specificity for the target, but had no effect on the circulation half-life of a targeting molecule/drug conjugate having different specificity. Compare FIGS. 5B and 3B, respectively. The reduction of circulation half-life may be attributable to retention of the targeting molecule/drug conjugate in the presence of an appropriate target.

IIA. Methods of Determining an Amount of Targeting Molecule in a Sample Comprising Targeting Molecule/Drug Conjugates

The present invention provides methods of determining an amount of targeting molecule in a sample comprising a plurality of targeting molecule/drug conjugates. In a particular aspect of the invention, the method comprises the steps of (a) providing a solid support comprising a surface to which a target is immobilized; (b) providing a sample comprising a plurality of targeting molecule/drug conjugates; (c) contacting the sample with the target immobilized to the surface of the solid support; and (d) detecting formation of a binding complex of (i) targeting molecules in the sample and (ii) the target at the surface of the solid support, wherein formation of the binding complex causes a measurable change in mass property of the solid support.

Representative samples that may be used in accordance with the disclosed methods include targeting molecule/drug conjugate preparations, i.e., a sample comprising a conjugation reaction between a targeting molecule and a drug, which may include conjugated targeting molecule, unconjugated targeting molecule, and free drug. Samples obtained from a subject following administration of antibodies to the subject may also be used, for example, blood, serum, or urine samples. The sample can comprise a minimal liquid volume, such as a sample less than about 100 μl, or less than about 50 μl, or less than about 25 μl, or less than about 10 μl, or less than about 5 μl. Larger sample volumes may be used to increase sensitivity. A sample may also comprise a liquid extract prepared from a tissue sample, such as a tumor. For example, a sample may be prepared from a squamous/adenomatous lung carcinoma (non-small-cell lung carcinoma), invasive breast carcinoma, colorectal carcinoma, gastric carcinoma, squamous cervical carcinoma, invasive endometrial adenocarcinoma, invasive pancreas carcinoma, ovarian carcinoma, squamous vesical carcinoma, choriocarcinoma, or other carcinomas of bronchi, breast, colon, rectum, stomach, cervix, endometrium, pancreas, ovaria, chorium, and seminal vesicles.

IIB. Methods of Determining an Amount of Drug in a Sample Comprising Targeting Molecule/Drug Conjugates

For determining an amount of drug in a sample, targeting molecule/drug conjugates are bound to a mass-sensing chip, and a drug binding agent that specifically binds to the drug moiety of the targeting molecule/drug conjugate is used to detect the conjugates. A drug binding agent can comprise an anti-drug antibody, or drug-binding fragment thereof. Additional representative binding agents include proteins, peptides, peptide mimetics, peptide nucleic acids (PNAs), ligands, or any other molecule that specifically binds to a drug moiety as described herein.

For example, the method may comprise the steps of (a) providing a solid support comprising a surface to which a first binding complex is immobilized, wherein the binding complex comprises (i) a target as described herein and (ii) a targeting molecule/drug conjugate bound to the target; (b) contacting an anti-drug antibody or drug-binding fragment thereof with the first binding complex immobilized at the surface of the solid support; and (c) detecting formation of a second binding complex of (i) the anti-drug antibody or drug-binding fragment thereof and (ii) the first binding complex at the surface of the solid support, wherein formation of the complex causes a measurable change in mass property of the solid support indicating an amount of targeting molecule/drug conjugate in the sample.

Alternatively, the method may comprise the steps of (a) providing a solid support comprising a surface to which an anti-drug antibody or drug-binding fragment thereof is immobilized; (b) contacting a sample comprising targeting molecule/drug conjugates with the anti-drug antibody or drug-binding fragment immobilized at the surface of the solid support; and (c) detecting a measurable change in mass property of the solid support indicating an amount of targeting molecule/drug conjugate in the sample.

An antibody that is used to detect the drug moiety of the targeting molecule/drug conjugate may be any antibody that shows specific binding, i.e., preferential binding to the drug when the drug is presented in a sample containing other antigens. The antibody may be polyclonal or monoclonal. Anti-drug antibodies having low off rates provide the greatest sensitivity. When using anti-drug antibodies having moderate off-rates, background corrections may be used to quantify targeting molecule/drug conjugates at reduced sensitivity.

Methods for preparing and characterizing anti-drug antibodies are well known in the art. See, e.g., Harlow & Lane, Antibodies: A Laboratory Manual, Cold Spring Harbor Laboratory, 1988. Additional techniques and reagents useful for generating and screening an antibody display library can be found in, for example, U.S. Pat. No. 5,223,409 and PCT International Application Publication Nos. WO 92/18619, WO 91/17271, WO 92/20791, WO 92/15679, WO 93/01288, WO 92/01047, WO 92/09690, and WO 90102809.

Briefly, a polyclonal antibody is prepared by immunizing an animal with an immunogen comprising a drug as described herein, and collecting antisera from that immunized animal. A wide range of animal species can be used for the production of antisera, for example rabbits, mice, rats, hamsters, guinea pigs, goats, and donkeys.

As is well known in the art, the immunogen may be coupled with a carrier, such as keyhole limpet hemocyanin (KLH) and serum albumins (e.g., BSA), to improve immunogenicity. Techniques for conjugating an immunogen to a carrier polypeptide are well known in the art and include glutaraldehyde, m-maleimidobencoyl-N-hydroxysuccinimide ester, carbodiimide and bis-biazotized benzidine. Immunogenicity of an immunogen can also be enhanced by the use of adjuvants, for example, complete Freund's adjuvant, incomplete Freund's adjuvants, and aluminum hydroxide adjuvant.

The amount of immunogen used for the production of polyclonal antibodies varies upon the nature of the immunogen, the animal used for immunization, and the administration route (e.g., subcutaneous, intramuscular, intradermal, intravenous, or intraperitoneal). The production of polyclonal antibodies is monitored by sampling blood of the immunized animal at various points following immunization. When a desired titer of antibody is obtained, the immunized animal is bled and the serum isolated and stored.

An anti-drug monoclonal antibody for use in the disclosed methods can be readily prepared through use of well-known techniques such as those exemplified in U.S. Pat. No. 4,196,265. For example, mice or rats are immunized with a drug for a sufficient period to obtain an immune response, and then spleen cells from the immunized animal are then fused with immortal myeloma cells. Fused cells are separated from the mixture of non-fused parental cells, for example, by the addition of agents to the culture media that block de novo nucleotide synthesis (e.g., aminopterin, methotrexate, and azaserine). Individual hybridomas are cultured and supernatants are tested for reactivity with the drug immunogen. The selected clones can be propagated indefinitely as a source of the monoclonal antibody.

By way of specific example, to produce an anti-drug antibody as described herein, mice are injected intraperitoneally with between about 1-200 μg of an antigen comprising a drug of a targeting molecule/drug conjugate. B lymphocyte cells are stimulated to grow by injecting the drug in association with an adjuvant such as complete Freund's adjuvant. As needed, mice are boosted by injection with a second dose of the drug mixed with incomplete Freund's adjuvant. A few weeks after the second injection, mice are tail bled and the sera titered by immunoprecipitation. The steps of boosting and titering are repeated until a suitable titer is achieved. The spleen of the mouse is removed, spleen lymphocytes are isolated, and myeloma cells are combined with the spleen lymphocytes under conditions appropriate for cell fusion. Fusion conditions include, for example, the presence of polyethylene glycol. Fused cells are separated from unfused myeloma cells by culturing in a selection medium such as HAT media (hypoxanthine, aminopterin, thymidine). The resultant hybridomas are screened for the production of anti-drug antibodies. Selected clones are cultured in high volumes to achieve suitable amounts of antibody. The antibodies may be purified by affinity chromatography or other methods, as is known in the art.

### EXAMPLES

The following examples have been included to illustrate modes of the invention. Certain aspects of the following examples are described in terms of techniques and procedures found or contemplated by the present co-inventors to work well in the practice of the invention. These examples illustrate standard laboratory practices of the co-inventors. In light of the present disclosure and the general level of skill in the art, those of skill will appreciate that the following examples are intended to be exemplary only and that numerous changes, modifications, and alterations may be employed without departing from the scope of the invention.

### Example 1

### Preparation of Antibody/Calicheamicin Conjugates

Gemtuzumab ozogamicin and inotuzumab ozogamicin are calicheamicin conjugates of the anti-CD33 and anti-CD22 antibodies, hP67.6 and G5/44, respectively. Gemtuzumab ozogamicin is the generic name for the marketed drug MYLOTARG® and is also referred to as hP67.6-AcBut-CalichDMH. The anti-CD22/calicheamicin conjugate, inotuzomab ozogamicin, also known as G5/44-AcBut-CalichDMH, is currently in phase I clinical trials. To obtain these conjugates, hP67.6 and G5/44 were linked to N-acetyl gamma calichemicin dimethyl hydrazide with the acid labile (4-(4′ acetylphenoxy)butanoic acid (AcBut) linker. Antibodies were loaded at a density of approximately 35 μg calicheamicin per mg hP67.6 and approximately 73 μg calicheamicin per mg G5/44. Anti-Lewis Y/calicheamicin and anti-5T4/calicheamicin conjugates were similarly prepared and used in the disclosed assays.

### Example 2

### Administration of Antibody/Calicheamicin Conjugates

The Ramos cell line (CRL-1923) was obtained from the American Type Culture Collection (ATCC). Ramos is a CD22⁺, CD33⁻ cell line derived from a human B-cell lymphoma. The cells were maintained in suspension cultures in RPMI1640 supplemented with 10 mM HEPES, 1 mM sodium pyruvate, 0.2% (w/v) glucose, 100 U/ml penicillin G sodium, 100 μg/ml streptomycin sulphate and 10% (v/v) fetal bovine serum.

Balb/c nude mice of 16 weeks old (Charles River Laboratories, Wilmington, Mass.) were irradiated with 400 rad gamma rays. Ramos cells (10⁷/200 μl) were injected in the right flank of each mouse. After 8 days, 10 mice with a tumor size of approximately 0.5 cm³ (±s=0.16) were selected. Four treatment groups were created: (1) tumor-bearing mice treated with hP67.6-AcBut-CalichDMH, (2) tumor-free mice treated with hP67.6-AcBut-CalichDMH, (3) tumor-bearing mice treated with G5/44-AcBut-CalichDMH, and (4) tumor-free mice treated with G5/44-AcBut-CalichDMH. Two days prior to administration of antibody/calicheamicin conjugates, a 5 μl blood sample was taken from each mouse. A single dose of 150 μl antibody/calicheamicin conjugate (3 μg calicheamicin per mouse) was injected into the lateral tail vein. Blood samples of exactly 5 μl were taken at 24, 48, 72, and 96 hours thereafter. To obtain reproducible small volume samples, the mice were kept under a heating lamp until tail veins became visible. The tail was disinfected with 70% isopropyl alcohol, and the lateral tail vein was ruptured with a needle. The resultant blood droplet was then aspirated with a capillary mounted to a micropipettor (Drummond of Broomall, Pa.) preset to an aspiration volume of 5 μl. This blood sample was immediately transferred to a test tube containing 195 μl of the following mixture: 0.01 M HEPES (pH 7.4), 0.15 M NaCl, 3 mM EDTA, 0.005% Surfactant P20 (HBS-EP buffer, available from Biacore of Uppsala, Sweden).

### Example 3

### Plasmon Resonance Sandwich Detection Assay

A plasmon resonance sandwich detection assay was developed to determine in a serum sample (1) an amount of targeting molecule and targeting molecule/drug conjugate in a sample, and (2) an amount of drug present in targeting molecule/drug conjugates of the same sample. The principle of this method is illustrated in FIG. 1. The assay allows for an evaluation of the clearance of targeting molecule/drug conjugate. The method does not discriminate between a reduction of drug on all the conjugate molecules and the generation of a fraction of unconjugated antibody.

The analyses described herein were performed on a BIACORE® instrument (Biacore International AB of Uppsala, Sweden) using antibody/calicheamicin conjugates. The detection system of this instrument relies upon the measurement of refractive index changes caused by the interaction of macromolecules on biosensor chips. See e.g., Johne et al., J. Immunol. Methods, 1993, 160(2):191-198; Karlson et al., J. Immunol. Methods, 1991, 145(1-2):229-240.

Antigens were immobilized to the surface of a CM5 biosensor chip at a density of 4000-9000 resonance units/flow cell. The chip was activated by the coupling reagent 1 -ethyl-3-(3-dimethylaminopropyl)-carbodiimide-HCl/N-hydroxysuccinimide at a flow rate of 5 μl/minute for 6 minutes, followed by addition of antigens. Lewis-BSA antigens were loaded by contacting the chip with 50 μg/ml protein in a solution of 10 mM sodium acetate (pH 4.0-4.5) at a flow rate of 5 μl/minute for 6 minutes. CD33 or CD22Fc were covalently linked to CM5 chips by contacting the chip to 0.1 mg/ml protein in a solution of 10 mM sodium acetate (pH 5) at a flow rate of 2 μl per minute for 30 minutes. The chip was then washed with HBS-EP containing 300 mM NaCl.

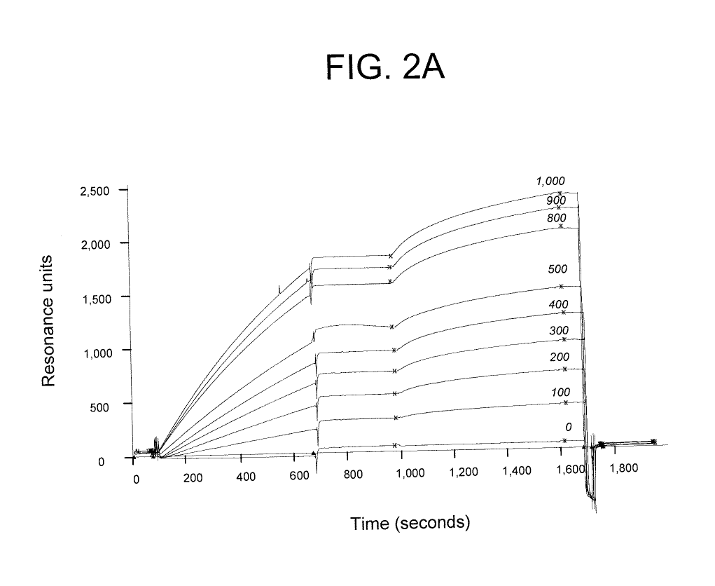

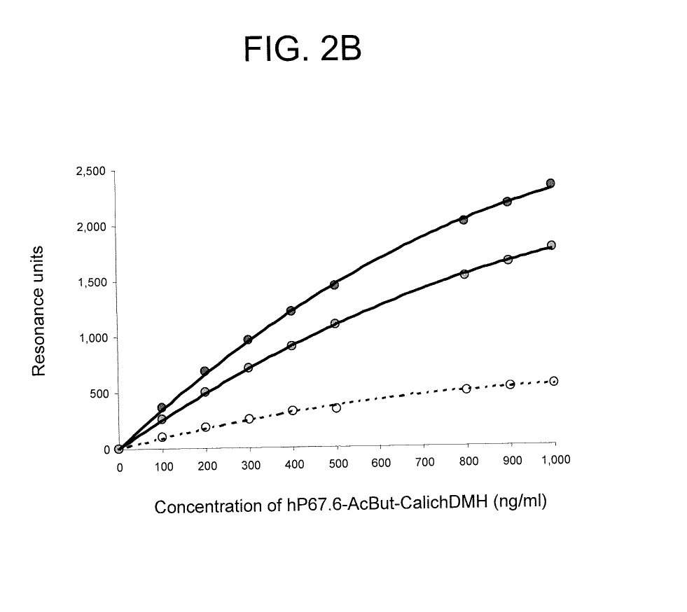

Following immobilization of the antigen on a CM5 chip, calibration curves were established for each antigen. As a representative result, FIGS. 2A-2B show the correlation between the concentration of standard samples and the number of resonance units upon binding of the anti-CD33/calicheamicin conjugate hP67.6-AcBut-CalichDMH. A correlation coefficient of approximately 1.0 allows for accurate determination of the total amount of antibody and the amount of calicheamicin bound to antibody. Using the calibration curves, the serum concentration of the antibody moiety of an antibody/drug conjugate was determined.

By thereafter contacting the chip with an anti-calicheamicin antibody, the amount of calicheamicin present in the serum sample was also determined. As demonstrated by the absence of a second response in FIG. 2B, unconjugated antibody at the same concentrations does not react to the anti-calicheamicin antibody. This result provides evidence for the specificity of the second response for the presence of calicheamicin on the antibody.

The response after binding of the conjugate to CD33 by itself (hP67.6-AcBut-CalichDMH) as well as followed by a secondary response (hP67.6-AcBut-CalichDMH+anti-calicheamicin) was linear (r²=0.9996 and r²=0.9994, respectively) for a concentration range of conjugate between 0 and 500 ng/ml. The difference of these responses (i.e., resonance units attributable to binding of anti-calicheamicin) is also linear (r²=0.9947) within this range. The regression coefficients of the quadratic equations of these functions were larger than 0.99 when a concentration range of 0 to 1000 ng/ml was used. Interpolation using a quadratic equation of resonance units plotted as a function of concentration allows for the accurate determination of antibody/drug conjugate concentration in a sample containing between 0 and 1000 ng/ml of antibody/drug conjugate.

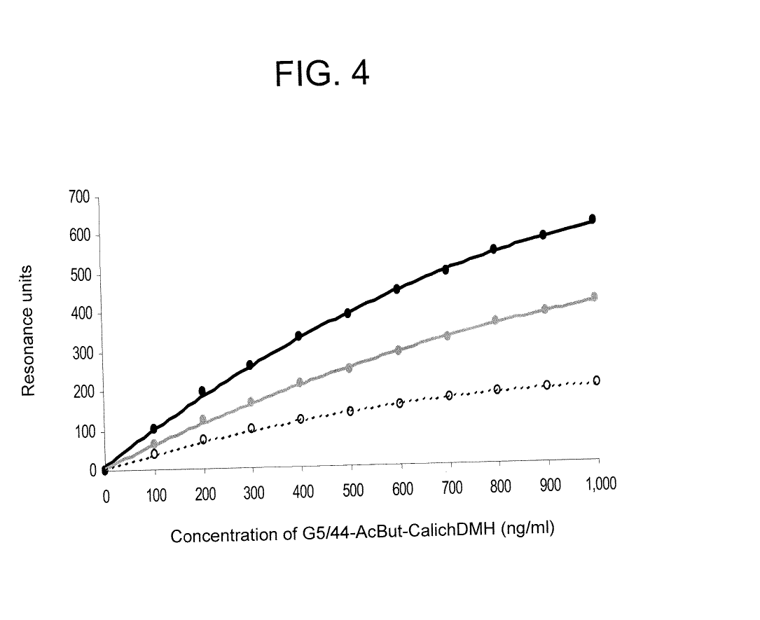

A similar strategy was used to establish the calibration curves depicting (1) the relationship between resonance units and the concentration of G5/44 anti-CD22 antibody and G5/44-AcBut-CalichDMH (see FIG. 4); and (2) the relationship between resonance units and the concentration of G193 anti-Lewis Y antibody and CMD193, a calicheamicin conjugate thereof. These relationships were also best described (r²>0.99) by a quadratic equation for a concentration range between 0 and 1000 ng/ml.

### Example 4

### Pharmacokinetic Properties of Anti-CD33/Calicheamicin Conjugates

The pharmacokinetic properties of hP67.6-AcBut-CalichDMH were determined in tumor-bearing and tumor-free mice. Five animals were used for each group. Tumor-bearing mice had an average body weight of 19 g (standard deviation=1 g) and had xenografted Ramos tumors with an average volume of 528 mm³ (standard deviation 102 mm³) Tumor-free mice had an average body weight of 20 g (standard deviation=1 g).

FIG. 3A shows the concentration of hP67.6-AcBut-CalichDMH in plasma of nude mice at various time points following intravenous injection of a single dose of antibody/drug conjugate. A dose of 3 μg calicheamicin was administered to each mouse. The dose of antibody as μg/kg body mass is indicated. The concentration of the antibody/drug conjugate in plasma was calculated by correcting for a normal hematocrit of 45%, and it was assumed that no antibody/drug conjugate was bound to the cell fraction. A 3 μg calicheamicin dose, which is provided as 86 μg antibody/drug conjugate having 35 μg calicheamicin per mg antibody, is administered in a blood volume of 1.5 ml (approximate blood volume of a 20 g mouse). Therefore, one would theoretically anticipate 105 μg/ml as a maximum concentration. Based upon a blood sample volume of 5 μl, the experimentally determined concentration of antibody/drug conjugate after 20 minutes was approximately 80 μg/ml.

The amounts of antibody/drug conjugate that were administered to each mouse varied depending on the actual body mass of the animal. Within a range of 4.1 to 4.5 mg antibody/drug conjugate per kg, the administered dose was not directly proportional to the maximum concentration of the conjugate in plasma. In addition, the data did not indicate that dose variation was responsible for variations in circulation half-life. An exceptionally high circulation half-life was observed in a single mouse that received a dose of 5 mg antibody/drug conjugate per kg.

The amount of hP67.6 conjugated to calicheamicin has a shorter circulation half-life than the unconjugated antibody. This is illustrated in FIG. 3C, which shows a consistently declining concentration of conjugated calicheamicin (response 2) as a fraction of the antibody-moiety of hP67.6-AcBut-CalichDMH (response 1). The reproducible reduction of total calicheamicin bound to antibody was not influenced by the presence of the CD22⁺ Ramos tumor.

### Example 5

### Pharmacokinetic Properties of Anti-CD22/Calicheamicin Conjugates

The pharmacokinetic properties of G5/44-AcBut-CalichDMH were determined in tumor-bearing and tumor-free mice. Three tumor-bearing mice had an average body weight of 19 g (standard deviation=1 g) and had xenografted Ramos tumors with an average volume of 1276 mm³ (standard deviation 398 mm³). Six tumor-free mice had an average body weight of 20 g (standard deviation=1 g). Administration of anti-CD22/calicheamicin conjugates and surface plasmon resonance assay were performed as described in Examples 2, 3, and 4.

Calibration curves depicting the relationship between resonance units and the concentration of the G5/44 antibody and G5/44-AcBut-CalichDMH conjugate are shown in FIG. 4. The relationship was best described (r>0.99) by a quadratic equation for a concentration range between 0 and 1000 ng/ml. See FIG. 4. As for unconjugated hP67.6, a response to free calicheamicin was not observed with unconjugated G5/44.

FIG. 5A shows the declining concentration of the antibody moiety of G5/44-AcBut-CalichDMH in plasma of tumor bearing and non-tumor bearing mice. Concentrations of the antibody moiety of G5/44-AcBut-CalichDMH (FIG. 5A) and of the amount of calicheamicin bound to G5/44 (FIG. 5B) declined faster in tumor-bearing mice. This was reflected in the decreased circulation half-life of G5/44-AcBut-CalichDMH. See Table I. The presence of a tumor that expresses the CD22 target enhanced the removal of the conjugate from plasma. The decline of the calicheamicin concentration as a function of time was identical in tumor bearing and non-tumor bearing mice (FIG. 5C), indicating that the presence of the tumor did not influence the release of calicheamicin from the antibody moiety of the conjugate.

TABLE I

| | −tumor | | +tumor |
|---|---|---|---|
| [symbol] | ²T | 55 ± 18* | 39 ± 21 |
| | AUC | 2,251 ± 406 | 997 ± 241 |
| | CL | 0.0012 ± 0.0002 | 0.0025 ± 0.0007 |
| | Vss | 5 ± 2 | 7 ± 4 |
| [symbol] | ²T | 29 ± 5.8 | 22.4 ± 6.3 |
| | AUC | 1,236 ± 233 | 681 ± 164 |
| | CL | 0.002 ± 0.000 | 0.004 ± 0.001 |
| | Vss | 4.6 ± 1.2 | 5.2 ± 0.9 |

AB = antibody moiety,
CM = calicheamicin bound to antibody,
²T = plasma half-life (h),
AUC = area under the curve (h * μg/ml),
CL = clearance (ml/min/kg),
Vss = volume distribution (ml/kg)

1. A method of determining an amount of targeting molecule and an amount of targeting molecule/drug conjugate in a sample comprising the steps of:
(a) providing a solid support comprising a surface to which a target is immobilized;
(b) providing a sample comprising a plurality of targeting molecule/drug conjugates;
(c) contacting the sample with the target immobilized to the surface of the solid support;
(d) detecting formation at the surface of the solid support of a first binding complex of (i) the targeting molecule and (ii) the target at the surface of the solid support, wherein formation of the first binding complex causes a first measurable change in mass property of the solid support indicating an amount of targeting molecule in the sample;
(e) contacting the first binding complex with a drug binding agent that specifically binds the drug of the targeting molecule/drug conjugate; and
(f) detecting formation at the surface of the solid support of a second binding complex of (i) the drug binding agent and (ii) the first binding complex, wherein formation of the second binding complex causes a second measurable change in mass property of the solid support indicating an amount of targeting molecule/drug conjugate in the sample.

2. The method of claim 1, wherein the target is expressed on cancer cells or on cells involved in an autoimmune response.

3. The method of claim 2, wherein the target expressed on cancer cells is 5T4, CD19, CD20, CD22, CD33, Lewis Y, HER-2, type I Fc receptor for immunoglobulin G (Fc gamma RI), CD52, epidermal growth factor receptor (EGFR), vascular endothelial growth factor (VEGF), DNA/histone complex, carcinoembryonic antigen (CEA), CD47, VEGFR2 (vascular endothelial growth factor receptor 2 or kinase insert domain-containing receptor, KDR), epithelial cell adhesion molecule (Ep-CAM), fibroblast activation protein (FAP), Trail receptor-1 (DR4), progesterone receptor, oncofetal antigen CA19.9, or fibrin.

4. The method of claim 1, wherein the targeting molecule is an antibody.

5. The method of claim 1, wherein the drug is calicheamicin.

6. The method of claim 1, wherein the drug binding agent is an antibody.

7. The method of claim 1, wherein the sample comprises a volume of about 5 μl or less.

8. The method of claim 1, wherein the sample is a blood sample.

9. A method of determining an amount of targeting molecule/drug conjugate in a sample comprising the steps of:
(a) providing a solid support comprising a surface to which a first binding complex is immobilized, wherein the binding complex comprises (i) a target and (ii) a targeting molecule/drug conjugate bound to the target;
(b) contacting a drug binding agent that specifically binds the drug of the targeting molecule/drug conjugate with the first binding complex immobilized at the surface of the solid support; and
(c) detecting formation of a second binding complex of (i) the drug binding agent and (ii) the first binding complex at the surface of the solid support, wherein formation of the complex causes a measurable change in mass property of the solid support indicating an amount of targeting molecule/drug conjugate in the sample.

10. The method of claim 9, wherein the target is expressed on cancer cells or on cells involved in an autoimmune response.

11. The method of claim 9, wherein the target expressed on cancer cells is 5T4, CD19, CD20, CD22, CD33, Lewis Y, HER-2, type I Fc receptor for immunoglobulin G (Fc gamma RI), CD52, epidermal growth factor receptor (EGFR), vascular endothelial growth factor (VEGF), DNA/histone complex, carcinoembryonic antigen (CEA), CD47, VEGFR2 (vascular endothelial growth factor receptor 2 or kinase insert domain-containing receptor, KDR), epithelial cell adhesion molecule (Ep-CAM), fibroblast activation protein (FAP), Trail receptor-1 (DR4), progesterone receptor, oncofetal antigen CA19.9, or fibrin.

12. The method of claim 9, wherein the targeting molecule is an antibody.

13. The method of claim 9, wherein the drug is calicheamicin.

14. The method of claim 9, wherein the drug binding agent is an antibody.

15. The method of claim 9, wherein the sample comprises a volume of about 5 μl or less.

16. The method of claim 9, wherein the sample is a blood sample.

17. The method of claim 9, wherein the amount of targeting molecule in the sample is determined by measuring a change in mass property of a solid support upon binding of targeting molecule/drug conjugates to a target immobilized at a surface of a solid support.

18. A method of determining an average amount of drug loading per targeting molecule in a sample of targeting molecule/drug conjugates comprising the steps of:
(a) providing a solid support to which targeting molecule/drug conjugates of a sample are bound;
(b) determining an amount of drug in the sample by measuring a change in mass property of a solid support upon binding of a drug binding agent that specifically binds the drug of the targeting molecule/drug conjugate to the targeting molecule/drug conjugates at the surface of the solid support; and
(c) calculating an average amount of drug per targeting molecule/drug conjugate by dividing the amount of drug of (b) by an amount of targeting molecule in the sample.

19. The method of claim 18, wherein the target is expressed on cancer cells or on cells involved in an autoimmune response.

20. The method of claim 18, wherein the target expressed on cancer cells is 5T4, CD19, CD20, CD22, CD33, Lewis Y, HER-2, type I Fc receptor for immunoglobulin G (Fc gamma RI), CD52, epidermal growth factor receptor (EGFR), vascular endothelial growth factor (VEGF), DNA/histone complex, carcinoembryonic antigen (CEA), CD47, VEGFR2 (vascular endothelial growth factor receptor 2 or kinase insert domain-containing receptor, KDR), epithelial cell adhesion molecule (Ep-CAM), fibroblast activation protein (FAP), Trail receptor-1 (DR4), progesterone receptor, oncofetal antigen CA19.9, or fibrin.

21. The method of claim 18, wherein the targeting molecule is an antibody.

22. The method of claim 18, wherein the drug is calicheamicin.

23. The method of claim 18, wherein the drug binding agent is an antibody.

24. The method of claim 18, wherein the sample comprises a volume of about 5 μl or less.

25. The method of claim 18, wherein the sample is a blood sample.

26. The method of claim 18, wherein the amount of targeting molecule in the sample is determined by measuring a change in mass property of a solid support upon binding of targeting molecule/drug conjugates to a target immobilized at a surface of a solid support.
